(19) United States
(12) Patent Application Publication
(10) Pub. No.: US 2019/0125575 A1
(43) Pub. Date: May 2, 2019

(54) ENHANCED MALE PROPHYLACTIC SHIELD

(71) Applicant: Matthew Alan Williams, Westland, MI (US)

(72) Inventor: Matthew Alan Williams, Westland, MI (US)

(21) Appl. No.: 16/177,409

(22) Filed: Oct. 31, 2018

Related U.S. Application Data
(60) Provisional application No. 62/580,368, filed on Nov. 1, 2017.

(51) Int. Cl. A61F 6/04

(52) U.S. Cl. CPC A61F 6/04; A61F 2006/047

(57) ABSTRACT

An enhanced male prophylactic shield includes a first portion and a second portion. The first portion defines a concavity for containing a scrotum. The second portion extends from the rim of the first portion and defines a hole through which a penis is extendable. Adhesive is mounted to the abdominal panel in a position to adhere to the abdomen of the user at a position superior to the penis.

### CROSS-REFERENCE TO RELATED APPLICATION

This application claims the benefit of U.S. provisional patent application No. 62/580,368, filed Nov. 1, 2017, and which is hereby incorporated by reference in its entirety.

### TECHNICAL FIELD

This disclosure relates to male sexual prophylactic shields.

### BACKGROUND OF THE INVENTION

Condoms are often employed to prevent the transmission of sexually transmitted diseases. A condom covers the male's penis, thereby preventing contact between the penis and the male's sexual partner and limiting bodily fluid transmission from the male to the male's partner. However, because the condom only covers the penis, other male body parts are still exposed and susceptible to contamination by contact with the male's partner or the partner's bodily fluids.

### SUMMARY OF THE INVENTION

An enhanced male prophylactic shield is provided. According to one aspect of the invention, the prophylactic shield comprises a material defining a scrotal pouch portion and an abdominal shield portion. The scrotal pouch portion is sized and shaped to at least partially contain the scrotum of a user. The abdominal shield portion extends from the scrotal pouch portion and defines an aperture. The aperture is sized and positioned such that the penis of the user is extendable therethrough. Adhesive is mounted to the abdominal shield portion in a position to adhere to the abdomen of the user at a position superior to the penis.

The prophylactic shield provided herein is configured to cover the user's scrotum, the region around the base of the penis, and at least the lower portion of the user's abdomen, and thus provides a barrier between these parts of the user and a sexual partner and/or the bodily fluids of a sexual partner. The prophylactic shield provides this additional coverage with less material than the prior art. Furthermore, the application of the shield is simplified compared to the prior art, which is important during romantic encounters. Moreover, the prophylactic shield is less obtrusive and more aesthetically pleasing compared to the prior art, which is particularly important during romantic encounters.

A corresponding method of use is also provided. The method includes possessing a male prophylactic shield having material defining a scrotal pouch portion and an abdominal shield portion. The scrotal pouch portion defines a cavity having only a single opening. The abdominal shield portion at least partially defines an aperture. The method also includes inserting a scrotum at least partially into the concavity through the opening, extending a penis through the aperture, and adhering the panel to an abdomen.

The above features and advantages and other features and advantages of the present disclosure are readily apparent from the following detailed description of the best modes for carrying out the disclosure when taken in connection with the accompanying drawings.

### BRIEF DESCRIPTION OF THE DRAWINGS

FIG. 1 is a schematic front view of a prophylactic shield applied to a human male;

FIG. 2 is a schematic, cross-sectional side view of the shield and male of FIG. 1;

FIG. 3 is a schematic, side view of the shield of FIGS. 1 and 2;

FIG. 4 is a schematic, front view of the shield of FIGS. 1-3;

FIG. 5 is a schematic, rear view of the shield of FIGS. 1-4;

FIG. 6 is a schematic, top view of the shield of FIGS. 1-5; and

FIG. 7 is a schematic, perspective view of the shield of FIGS. 1-6.

### DESCRIPTION OF THE PREFERRED EMBODIMENTS

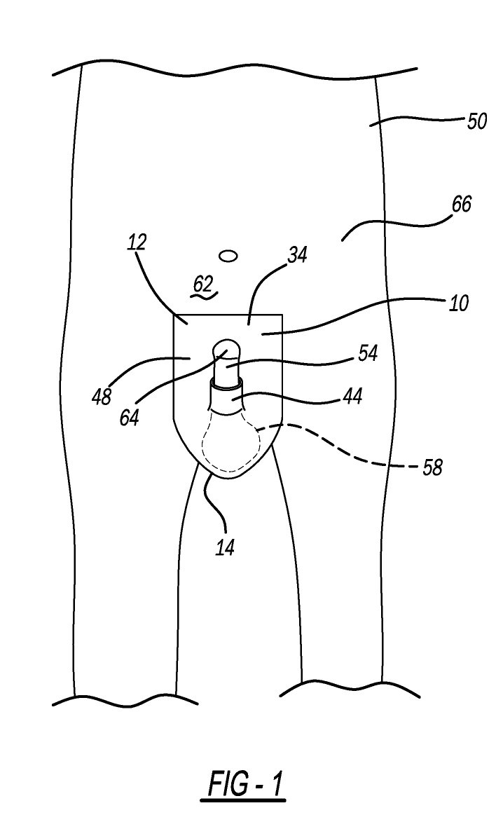

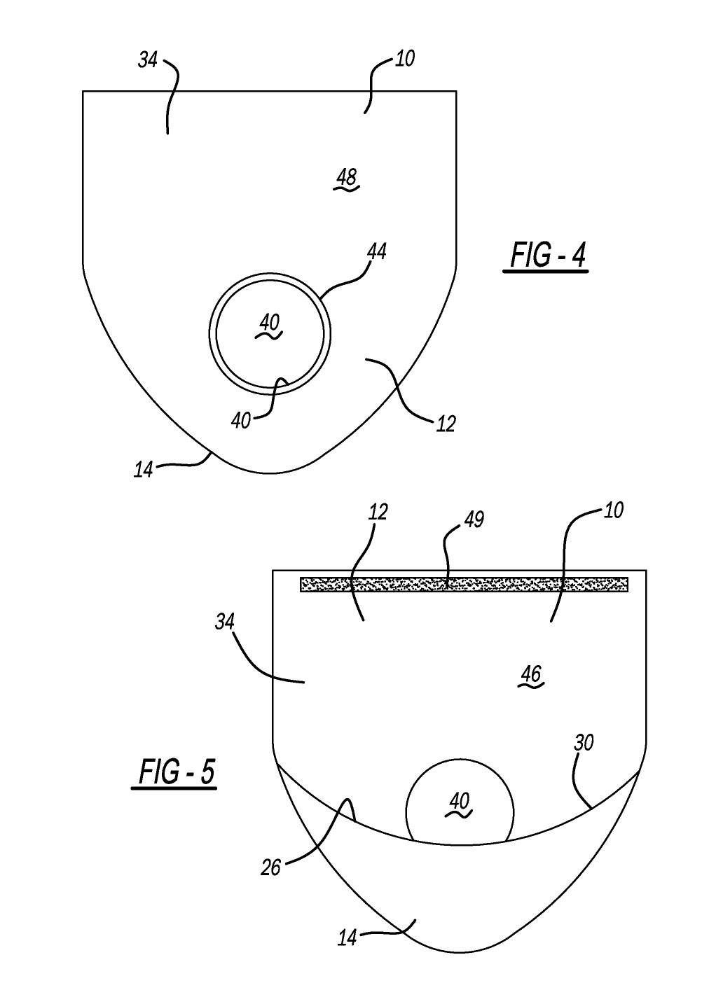

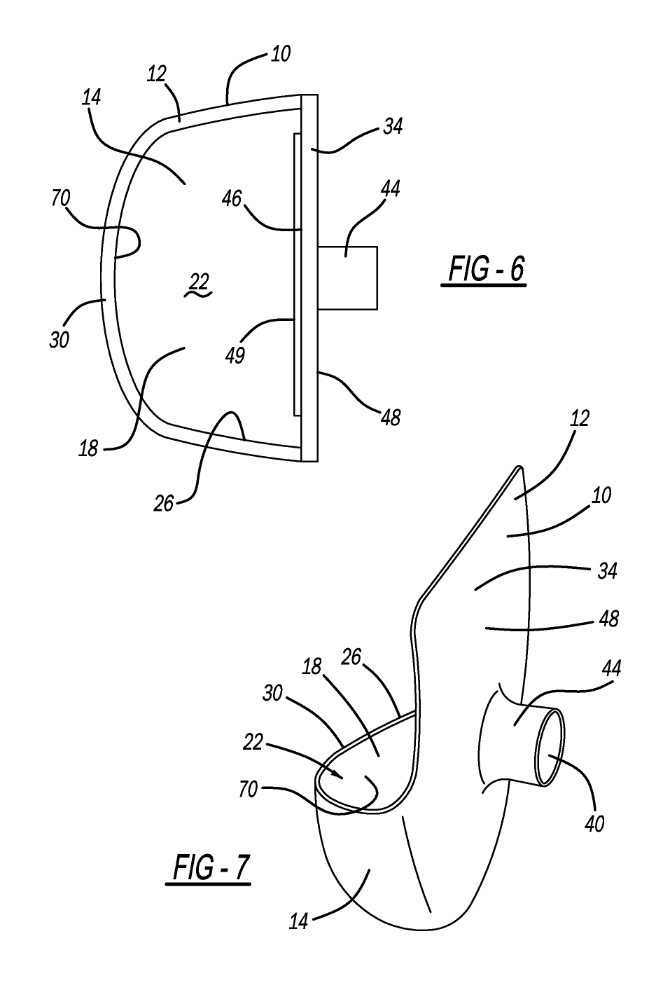

Referring to FIGS. 1-7, wherein like reference numbers refer to like components throughout, an enhanced male prophylactic shield 10 is schematically depicted. The shield 10 in the embodiment depicted is formed by a material such as latex. The shield 10 in the embodiment depicted is formed from a single piece 12 of material, but may be formed by multiple pieces of material fastened together within the scope of the claims. Other relatively thin, flexible, non-porous, non-adsorbent, and impermeable materials may be employed within the scope of the claims, such as, but not limited to, latex, polyurethane, polyisoprene, nitrile, and silicone.

The shield 10 includes a first portion, i.e., a scrotal pouch portion 14. The scrotal pouch portion 14 is characterized by a concave surface 18 defining a chamber or concavity 22 having an opening 26 defined by an edge 30 of the piece 12. Edge 30 forms a rim around a portion of the opening 26.

The shield 10 includes a second portion, i.e., abdominal shield portion 34 formed by the material 12. The abdominal shield portion 34 extends from the scrotal pouch portion 14. More specifically, in the embodiment depicted, the abdominal shield portion 34 is generally rectangular, and at least partially defines a hole or aperture 40. As shown in FIGS. 3 and 7, in the embodiment depicted, the edge 30 defining the opening 26 is generally coplanar, and thus the opening 26 is generally situated about a plane. The abdominal shield portion 34 is generally perpendicular to the plane. In the embodiment of FIGS. 1-7, the shield 10 includes a third portion, i.e., penile extension portion 44, that cooperates with the abdominal shield portion 34 to define the aperture 40. Penile extension portion 44, and the aperture 40, may, for example, be cylindrical or frusto-conical. Aperture 40 extends from one side 46 of the abdominal shield portion 34 to the opposite side 48 of the shield portion 34 and through the penile extension portion 44.

An adhesive 49 is applied and attached to side 46 of the abdominal shield portion 34. Those skilled in the art will recognize a variety of adhesives that may be employed to adhere the abdominal shield portion 34 to human skin in a manner that is secure yet easily removed by low peal or shear force. In one embodiment, the adhesive 49 is an atraumatic silicone gel adhesive.

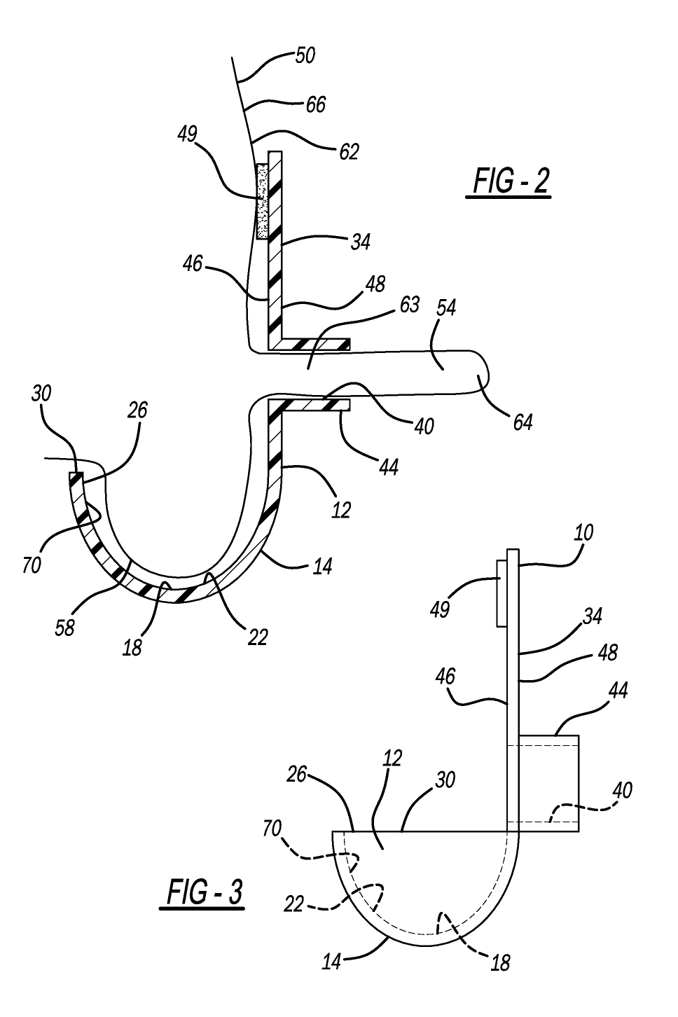

Referring specifically to FIGS. 1 and 2, the shield 10 is shown in use on a male human body 50. The body 50 includes a penis 54 and scrotum 58, as understood by those skilled in the art. A method of use for the shield 10 includes possessing the shield 10, and then inserting at least a portion of the scrotum 58 into the concavity 22 via opening 26. In the embodiment depicted, substantially all of the scrotum 54 is disposed within the concavity 22. The method also includes inserting or extending the penis 54 into and through the aperture 40 until the second portion 34 contacts the skin 62 surrounding the base of the penis 54. The penis 54 extends through the aperture 40 and, correspondingly, the penile extension portion 44, such that penile extension portion 44 contacts and surrounds a portion of the penis 54 near the proximal end of the penis 54. The distal end of the penis 54 may then be covered by a traditional condom.

The abdominal shield portion 34 extends in the superior direction (i.e., upward if the body 50 is standing upright) and the adhesive 49 on the shield portion 34 is placed in contact with the skin 62 on or near the abdomen 66, or at least superior to the base of the penis 54.

The prophylactic shield 10 is retained on the body 50 solely by the adhesive 49; by the interaction between the penis 54 and the material defining the aperture 40; and by the interaction between the scrotum 58 and the concave surface 18. More specifically, and with reference to FIG. 2, a portion 70 of surface 18 and edge 30 are directly and immediately posterior of the scrotum 58. Interaction between portion 70 and the scrotum 58 retains the scrotal pouch portion 14 in place and limits movement of shield 10 in the anterior or forward direction. This mounting arrangement eliminates the need for cumbersome straps to attach the shield 10 to the body, thereby minimizing material usage, enhancing aesthetics, and facilitating rapid and simple application. In the embodiment depicted, the shield 10 includes only the scrotal pouch portion 14, the second portion 34, and the third portion 44.

It should be noted that, in an alternative embodiment, shield 10 does not include the penile extension portion 44, and aperture 40 would simply extend through the abdominal shield portion 34. As used herein, “abdomen” and “abdominal” may include frontal regions of the pelvis superior to the penis.

While the best modes for carrying out the invention have been described in detail, those familiar with the art to which this invention relates will recognize various alternative designs and embodiments for practicing the invention within the scope of the appended claims.

1. A male prophylactic shield for use on a male human having a penis and scrotum, the shield comprising:
material having a first portion and a second portion;
said first portion including a surface defining a concavity, and an edge at least partially defining an opening of the concavity;
said second portion extending from, and connected to, the first portion, having a first side and a second side, and at least partially defining an aperture that extends from the first side to the second side; and
adhesive affixed to the first side of the second portion.

2. The male prophylactic shield of claim 1, wherein the material is sized and shaped such that, when the scrotum is at least partially within the concavity and the penis extends through the aperture, at least a portion of the surface defining the concavity is directly and immediately posterior of the scrotum.

3. The male prophylactic shield of claim 2, wherein the material has a third portion that extends from the second portion and that cooperates with the second portion to define the aperture.

4. The male prophylactic shield of claim 3, wherein the third portion and the aperture are cylindrical or frusto-conical.

5. The male prophylactic shield of claim 2, wherein the material is non-porous and non-adsorbent.

6. The male prophylactic shield of claim 2, wherein the material is selected from the group consisting of latex, polyurethane, polyisoprene, nitrile, and silicone.

7. The male prophylactic shield of claim 1, wherein the first portion is characterized by the single opening.

8. The male prophylactic shield of claim 1, wherein the second portion is generally perpendicular to the edge.

9. A male prophylactic shield for a human body having a penis, a scrotum, and an abdomen, the shield comprising:
material defining a first portion and a second portion;
wherein the first portion defines a concavity;
wherein the second portion at least partially defines an aperture;
adhesive being affixed to the second portion;
wherein the male prophylactic shield is configured such that shield is positionable with respect to the body so that the concavity at least partially contains the scrotum, a portion of the first portion is directly and immediately posterior of the scrotum, the penis extends through the aperture, and the adhesive is in contact with the abdomen.

10. The male prophylactic shield of claim 9, wherein the interaction between the first portion and the scrotum, the interaction between the material defining the aperture and the penis, and the interaction between the adhesive and the abdomen are sufficient to attach the male prophylactic shield to the human.

11. The male prophylactic shield of claim 9, wherein the material defines a third portion that cooperates with the second portion to define the aperture; and
wherein the interaction between the first portion and the scrotum, the interaction between the penis and the third portion, and the interaction between the adhesive and the abdomen are sufficient to attach the male prophylactic shield to the human.

12. The male prophylactic shield of claim 9, wherein the first portion defines an opening to the concavity situated about a plane; and
wherein the second portion is generally perpendicular to the plane.

13. A method comprising:
possessing a male prophylactic shield having material defining a scrotal pouch portion and an abdominal shield portion, the scrotal pouch portion defining a cavity having only a single opening, the abdominal shield portion at least partially defining an aperture;
inserting a scrotum at least partially into the concavity through the opening such that at least a portion of the scrotal pouch portion is immediately and directly posterior of the scrotum;
extending a penis through the aperture; and
adhering the panel to an abdomen.

14. The method of claim 13, wherein the material has a penile extension portion that cooperates with the abdominal shield portion to further define the aperture; and
wherein the method further includes extending the penis through the penile extension portion.

15. The method of claim 14, wherein the aperture is cylindrical or frusto-conical.

16. The method of claim 14, wherein the material is non-porous and non-adsorbent.

17. The method of claim 16, wherein the material is selected from the group consisting of latex, polyurethane, polyisoprene, nitrile, and silicone.
